SEQUENCE LISTING (3 sequences)

SEQ ID NO: 1 - DNA, 1499 bp, Pseudomonas migulae
  source 1..1499: mol_type = genomic DNA
aagttttttattcagctcagattgaacgctggcggcaggcctaacacatgcaagtcgagcggtagagagaagcttgcttctcttgagagcggcggacgggtgagtaatgcctaggaatctgcctggtagtgggggataacgtccggaaacggacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccagaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaaggggggaaccc

SEQ ID NO: 2 - DNA, 18 bp, synthetic construct
  source 1..18: mol_type = other DNA
ggattagataccctggta

SEQ ID NO: 3 - DNA, 20 bp, synthetic construct
  source 1..20: mol_type = other DNA
ccgtcaattcmtttragttt

(19) United States
(12) Patent Application Publication
(10) Pub. No.: US 2024/0017310 A1
(43) Pub. Date: Jan. 18, 2024

(54) NOVEL MICROORGANISM FOR POLYSTYRENE BIODEGRADATION

(71) Applicants: Hyundai Motor Company, Seoul (KR); Kia Corporation, Seoul (KR); INDUSTRY FOUNDATION OF CHONNAM NATIONAL UNIVERSITY, (US)

(72) Inventors: Chae Hwan Hong, Seoul (KR); Young Ho Choi, Seongnam (KR); Soo Jin Yeom, Gwangju (KR); Won Seok Chi, Gwangju (KR)

(21) Appl. No.: 18/065,810

(22) Filed: Dec. 14, 2022

(30) Foreign Application Priority Data
Jul. 14, 2022 (KR) 10-2022-0086650

(51) Int. Cl. B09B 3/60; C12N 1/20

(52) U.S. Cl. CPC B09B 3/60; C12N 1/205; C12R 2001/38

(57) ABSTRACT

Disclosed are a composition including a Pseudomonas sp. strain having excellent plastic degradation activity and the use thereof. The Pseudomonas sp. Strain may include Pseudomonas sp. JNU 01 (Accession No: KCTC 14861BP).

### CROSS-REFERENCE TO RELATED APPLICATION

This application claims under 35 U.S.C. § 119(a) the benefit of priority to Korean Patent Application No. 10-2022-0086650 filed on Jul. 14, 2022, the entire contents of which are incorporated herein by reference.

### SEQUENCE LISTING

The contents of the sequence listing text file named “DP-2022-0141_sequence list.xml”, which was created on Dec. 8, 2022 and is 5,047 bytes in size, are incorporated herein by reference in its entirety.

### TECHNICAL FIELD

The present disclosure relates to a composition including an isolated microorganism having plastic degradation activity and the use thereof. The composition may have an activity of degrading plastic, particularly polystyrene.

### BACKGROUND

Waste includes a variety of materials such as metals, glass, and plastics. Plastics exist in far larger amounts than metals in both industrial and household wastes, and thus their value exceeds metals in economic terms. Meanwhile, due to the depletion of petroleum, which is the main raw material of plastics, the need to recycle plastics discharged as wastes is increasing.

For plastic recycling, various types of plastics are usually used in one product, and various types of plastics are collected at once and recycled. For this reason, completely maintaining the purity of each recycled plastic material as a single material is difficult because other recycled plastic materials act as impurities. As a result, the quality of each recycled plastic material is inferior, and thus the economic value thereof is estimated to be about 34% less than that of a new plastic material.

In particular, it is difficult to economically separate the mixed waste plastics contained in the household waste by properties, and thus there is an urgent need to develop technology for sorting the mixed waste plastics. A plastic recycling process largely consists of a pre-treatment process and a recycling process. In the pre-treatment process, the mixed waste plastics are separated and sorted by properties. A wet flotation method is mainly used to separate and sort the mixed waste plastics by properties. A large amount of water is required for wet flotation, and even after wet flotation, each plastic material is recycled as low-grade plastic because the content of other materials therein is about 2%.

Meanwhile, technology of treating plastics contained in wastewater or wastes using microorganisms has recently been developed. For example, in the related art, the novel microorganism Klebsiella pneumoniae CJ-PVA a (accession number KFCC-11126) that shows good growth under aerobic conditions and has an improved ability to degrade polyvinyl alcohol, and a method of treating wastewater containing polyvinyl alcohol using the novel microorganism.

In addition, a new strain of Microbacterium barkeri LC (accession number KCCM 10507) has been reported and a method of biologically degrading polyvinyl alcohol using the same.

However, studies on effective stains that degrade polystyrene plastic are insufficient and these studies are necessary.

### SUMMARY

In preferred aspects, provided are an isolated microorganism capable of degrading polystyrene plastic, compositions inducing the same, and a method of degrading a plastic using the same.

In preferred aspects, provided is a composition including an isolated Pseudomonas sp. strain having excellent plastic degradation activity and a microbial agent containing the same.

In an aspect, provided an isolated Pseudomonas migulae sp. JNU 01 (Pseudomonas sp. JNU 01) strain deposited under accession number KCTC 14861BP.

In an aspect, provided is a method of degrading a plastic using one or more of the Pseudomonas sp. JNU 01 strain, a lysate thereof, a culture thereof, a fermentation product thereof, and an extract of the strain, lysate, culture or fermentation product.

In an aspect, provided is a composition for plastic degradation including isolated one or more of the Pseudomonas sp. JNU 01 strain, a lysate thereof, a culture thereof, a fermentation product thereof, and an extract of the strain, lysate, culture or fermentation product.

Objects of the present disclosure are not limited to the above-mentioned objects. The objects of the present disclosure will become clearer from the following description, and will be realized by means and combinations thereof set forth in the claims.

In order to achieve the above objects, the following solutions are provided.

Provided is an isolated Pseudomonas sp. strain having plastic degradation activity. The plastic may include one or more of polyethylene terephthalate (PET), polyvinyl chloride (PVC), polystyrene (PS), polypropylene (PP) and polyethylene (PE).

The strain may be the one isolated from soil including plastic waste and can be cultured after inoculation into a medium containing polystyrene as a sole carbon source. For example, the soil was collected from soil contaminated with waste around Chonnam National University and around old cattle shed in Jangpyeong-myeon, Jangheung-gun, Jeollanam-do, Korea.

The strain may have polystyrene (PS) degradation activity.

The strain has a 16S rRNA including the nucleotide sequence of SEQ ID NO: 1.

The strain includes Pseudomonas sp. JNU 01.

Particularly, the strain includes Pseudomonas sp. JNU 01 deposited under accession number KCTC 14861BP.

Also provided is a microbial agent including one or more of the isolated strain, a lysate thereof, a culture thereof, a fermentation product thereof, and an extract of the strain, lysate, culture or fermentation product.

Further provided is a method of degradating plastic, and the method includes using one or more of the isolated strain according to one aspect of the present disclosure, a lysate thereof, a culture thereof, a fermentation product thereof, and an extract of the strain, lysate, culture or fermentation product.

The plastic may include polystyrene.

In addition, provided is a composition for plastic degradation including one or more of the isolated strain, a lysate thereof, a culture thereof, a fermentation product thereof, and an extract of the strain, lysate, culture or fermentation product.

The isolated strain may degrade plastic including polystyrene.

The strain and a composition including the same may provide an excellent plastic (e.g., polystyrene) degradation effect.

Other aspects of the invention are disclosed infra.

### BRIEF DESCRIPTION OF THE DRAWINGS

FIG. 1 shows a phylogenetic tree of the strain in an exemplary embodiment of the present disclosure.

FIG. 2 shows the results of Experimental Example 2 of the present disclosure.

FIG. 3 shows the results of Experimental Example 3 of the present invention. The photograph on the left of FIG. 3 shows a pure polystyrene (PS) film as a control, and the photograph on the right shows a pure polystyrene (PS) film treated with Pseudomonas sp. JNU 01.

FIGS. 4A and 4B show the results of Experimental Example 4.

### DETAILED DESCRIPTION

The objects, other objects, features and advantages of the present disclosure will become apparent with reference to the embodiments described below in conjunction with the accompanying drawings. However, the present disclosure is not limited to the embodiments disclosed below and may be embodied in a variety of different forms. Rather, these embodiments are provided so that this disclosure will be thorough and complete, and will fully convey the scope of the present disclosure to those skilled in the art.

In the present specification, it should be understood that terms such as “include” and “have” are intended to denote the existence of mentioned characteristics, numbers, steps, operations, components, parts, or combinations thereof, but do not exclude the probability of existence or addition of one or more other characteristics, numbers, steps, operations, components, parts, or combinations thereof.

Unless otherwise indicated, all numbers, values, and/or expressions referring to quantities of ingredients, reaction conditions, polymer compositions, and formulations used herein are to be understood as modified in all instances by the term “about” as such numbers are inherently approximations that are reflective of, among other things, the various uncertainties of measurement encountered in obtaining such values. Further, unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the present specification, where a range is stated for a parameter, it will be understood that the parameter includes all values within the stated range, inclusive of the stated endpoints of the range. For example, a range of 5 to 10 will be understood to include the values 5, 6, 7, 8, 9, and 10, as well as any sub-range within the stated range, such as to include the sub-range of 6 to 10, 7 to 10, 6 to 9, 7 to 9, etc., and inclusive of any value and range between the integers which is reasonable in the context of the range stated, such as 5.5, 6.5, 7.5, 5.5 to 8.5 and 6.5 to 9, etc. For example, a range of 10% to 30% will be understood to include the values 10%, 11%, 12%, 13%, etc., and all integers up to and including 30%, as well as any sub-range within the stated range, such as to include the sub-range of 10% to 15%, 12% to 18%, 20% to 30%, etc., and inclusive of any value and range between the integers which is reasonable in the context of the range stated, such as 10.5%, 15.5%, 25.5%, etc.

Hereinafter, the present disclosure will be described in detail.

In Experimental Example 2 according to an exemplary embodiment of the present disclosure, the growth curve of Pseudomonas sp. JNU 01 grown using polystyrene powder as a sole carbon source was measured.

As a result of measuring the growth curve of the strain of the present disclosure using a sole carbon source and the polystyrene degradation activity thereof, the optical density (OD) value of each sample changed for 12 days when the strain was cultured in a flask containing polystyrene and 10 mL of liquid medium. Each line was distinguished by the concentration of polystyrene: 2 mg/ml (blue), 6 mg/ml (green), 10 mg/ml (red).

Hereinafter, various aspects of the present disclosure will be described.

One aspect of the present disclosure provides a Pseudomonas sp. strain having plastic degradation activity.

The plastic may include any one or more of polyethylene terephthalate (PET), polyvinyl chloride (PVC), polystyrene (PS), The strain may be isolated from soil including or mixed with plastic waste and grows after inoculation into a medium containing polystyrene as a sole carbon source. The soil was collected from soil contaminated with waste around Chonnam National University and around old cattle shed in Jangpyeong-myeon, Jangheung-gun, Jeollanam-do, Korea.

The strain has polystyrene (PS) degradation activity.

The strain has a 16S rRNA including the nucleotide sequence of SEQ ID NO: 1.

The strain includes Pseudomonas sp. JNU 01.

For example, the strain may include the strain deposited under accession number KCTC 14861BP.

Another aspect of the present disclosure provides a microbial agent including one or more of the isolated strains, a lysate thereof, a culture thereof, a fermentation product thereof, and an extract of the strain, lysate, culture or fermentation product.

Still another aspect of the present disclosure provides a method of degradating plastic and the method may include using one or more of the isolated strains, a lysate thereof, a culture thereof, a fermentation product thereof, and an extract of the strain, lysate, culture or fermentation product.

The plastic to be degraded may include polystyrene.

Yet another aspect of the present disclosure provides a composition for plastic degradation including one or more of the isolated strains according to one aspect of the present disclosure, a lysate thereof, a culture thereof, a fermentation product thereof, and an extract of the strain, lysate, culture or fermentation product.

The plastic to be degraded may include polystyrene.

### Example

Hereinafter, the configuration and effects of the present disclosure will be described in more detail with reference to Examples and Experimental Examples. These Examples and Experimental Examples are only for illustrating the present disclosure, and the scope of the present disclosure is not limited by these Examples and Experimental Examples.

### Example 1. Isolation of JNU 01 Strain

Soil mixed with plastic waste was collected at Chonnam National University (35° 10′50″N, 126° 54′23″E, Gwangju, Korea). About 5 g of the soil sample was mixed with 100 mL of PBS buffer and then filtered through Whatman paper (pore size: 11 μm). The filtered solution was smeared on a solid basal salt medium (BSM) containing 1 g/L of polystyrene powder and cultured at a temperature of 28° C. for 7 days. The strain cultured on the polystyrene-containing medium was streaked on a solid BSM containing 1 g/L of polystyrene and separated, and then whether the strain was subcultured was examined. Then, the cultured bacteria were inoculated into a liquid medium containing 1 g/L of polystyrene powder and cultured at 28° C. and 200 rpm, and the optical density (OD) thereof was measured.

### Example 2. Identification of JNU 01 Strain

1) PCR Profiles

The selected strain was obtained by streaking on a NB solid medium plate (3.0 g beef extract, 5.0 g peptone and 15.0 g agar in 1 L of deionized water, pH 6.8).

InstaGene™ Matrix (BIO-RAD, CA, USA) was used to extract genomic DNA from the strain, and 16sRNA analysis was performed using the DNA isolated from the strain as a template to identify the bacterial species. For the PCR reaction, universal primers 785F (GGATTAGATACCCTGGTA; SEQ ID NO: 2) and 907R (CCGTCAATTCMTTTRAGTTT; SEQ ID NO: 3) were used (Macrogen, Daejeon, South Korea). The PCR reaction was performed using 20 ng of genomic DNA as a template, and the reaction was performed at a volume of 30 μl using EF-Taq (Solgent, Daejeon, South Korea). The PCR reaction was performed under the following conditions: a reaction for reactivation of Taq polymerase at 95° C. for 2 min; 35 cycles, each consisting of 95° C. for 1 min, 55° C. for 1 min, and 72° C. for 1 min; and then a reaction at 72° C. for 10 min. The PCR amplification product was purified using a multiscreen filter plate (Millipore Corp., MA, USA). Sequencing was performed using a PRISM BigDye Terminator v3.1 Cycle sequencing kit. Hi-Di formamide (Applied Biosystems, CA, USA) was added to the DNA sample containing the extension reaction product. The mixture was incubated at 95° C. for 5 minutes, incubated on ice for 5 minutes, and then analyzed using an ABI Prism 3730XL DNA analyzer (Applied Biosystem, CA, USA).

2) 16S rRNA Sequencing

For identification of the strain, the 16s rRNA of the strain was sequenced, and the homology with other standard strains registered in the Genbank database on the BLAST of NCBI was determined. The 16S rDNA sequence of the strain is set forth in SEQ ID NO: 1.

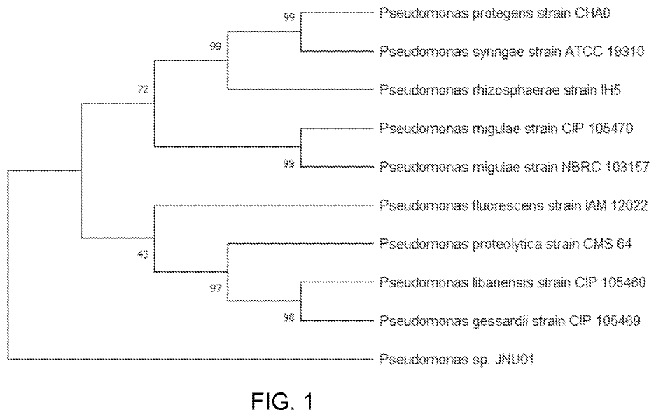

The relationship between 16s rRNA genes in the Genbank database was analyzed using the MUSCLE algorithm of Molecular Evolutionary Genetics Analysis Version X 11 (MEGA X 11.0.11). In addition, homology between strains was analyzed using MEGA X 11 to obtain a phylogenetic tree. The phylogenetic tree is shown in FIG. 1.

Accordingly, the present inventors identified the strain as a novel Pseudomonas sp. strain. The identified strain was named Pseudomonas sp. JNU01 and deposited with the Korean Collection for Type Cultures (KCTC) of the Korea Research Institute of Bioscience and Biotechnology on Feb. 11, 2022 under accession number KCTC 14861BP.

The following experiment was performed on the deposited JNU 01 strain.

### Experimental Example 1. Material Preparation

(1) Chemicals and Medium Composition

Nutrient broth (NB) medium (5.0 g peptone, 3.0 g/L beef extract), basal salt medium (BSM) (12.8 g Na₂HPO₄·7H₂O, 3 g KH₂PO₄, 0.5 g NaCl, 1 g NH₄Cl, 2 mM MgSO₄, 0.1 mM CaCl₂), per liter), styrene (ReagentPlus®, containing 4-tert-butylcatechol as stabilizer, ≥99%) and 2,2′-azobisisobutyronitrile (AIBN, 98%) were purchased from Sigma-Aldrich (St. Louis, MO, USA).

Methanol (MeOH)), DMF (N,N′-dimethylformamide) and THF (tetrahydrofuran (HPLC grade)) were purchased from Duksan (Ansan, South Korea). All chemicals and solvents were used without further purification.

(2) Synthesis of Polystyrene Powder and Film

Polystyrene was synthesized by free radical polymerization. In a 50-mL flask, 6.0 g of styrene was dissolved in 50 mL of a DMG (ethylene glycol dimethyl ether) solvent at room temperature. Thereafter, an azobisisobutyronitrile (AIBN) initiator was added to the styrene solution and vigorously stirred until the AIBN was completely dissolved.

The mixture was left in nitrogen gas (99.999%) for 30 minutes to remove oxygen. Through this process, it was possible to increase the time of radical polymerization by creating an inert atmosphere. The polymerization was carried out with slow stirring at a temperature of 90° C. for 16 hours. The polymerization product was precipitated in methanol and washed. The resultant was centrifuged at high speed (e.g., 10,000 rpm) and then washed with methanol. This washing step was repeated several times until the unreacted monomers and residues were completely removed. The polystyrene was dried under vacuum at 60° C. to remove the remaining solvent.

A polystyrene film was prepared by solvent casting. Polystyrene powder (0.3 g) was dissolved in THF solvent (6 mL) to prepare 0.5 wt % polystyrene solution. The polystyrene solution was stirred for several hours to induce complete polymer decomposition, thus preparing a homogeneous solution. The polystyrene solution was poured into a glass Petri dish, and then covered with a perforated aluminum foil to slow the evaporation rate of the solvent in the glass Petri dish.

The glass Petri dish covered with the aluminum foil was placed in an oven at a temperature of 50° C. overnight. After the solvent was completely evaporated, a transparent film was formed. The polystyrene film was easily peeled off from the substrate by pouring deionized water into the glass Petri dish. The separated polystyrene film was dried in an oven at a temperature of 50° C. for a while to remove the remaining volatile solvent. Finally, the dried polystyrene film was stored in a vacuum oven at 60° C. to remove residual moisture.

### Experimental Example 2. Bacterial Cell Growth in Polystyrene Medium

(1) Experimental Method

The JNU 01 strain selected in the above Example was inoculated into a liquid nutrient medium, and then cultured in a shaking incubator overnight at a temperature of 28° C. and 200 rpm. In order to remove the remaining carbon source, the culture containing the bacteria was centrifuged at 3,800 rpm for 20 minutes and then washed with basal salt medium (BSM).

The washed bacteria were inoculated onto a BSM containing polystyrene powder and cultured in a shaking incubator for 14 days at a temperature of 28° C. and 200 rpm.

The concentration of polystyrene powder for each flask was 2, 6 or and 10 mg/ml. Optical density (OD) was measured with a UV-Vis spectrophotometer (SHIMADZU, Kyoto, Japan).

(2) Experimental Results: Measurement of Growth Curve and Polystyrene Degradation Efficiency of Pseudomonas sp. JNU01

To confirm the growth of Pseudomonas sp. JNU01 on the BSM containing polystyrene, an increase in the optical density of the flask containing the BSM containing polystyrene as a carbon source was measured.

It was visually confirmed that, when the microorganism was not inoculated onto the BSM containing polystyrene, the medium was transparent.

The degradation and utilization of polystyrene by Pseudomonas sp. JNU01 was studied while observing cell growth on BSM media containing polystyrene at various concentrations (2 g/L, 6 g/L and 10 g/L) for 12 days.

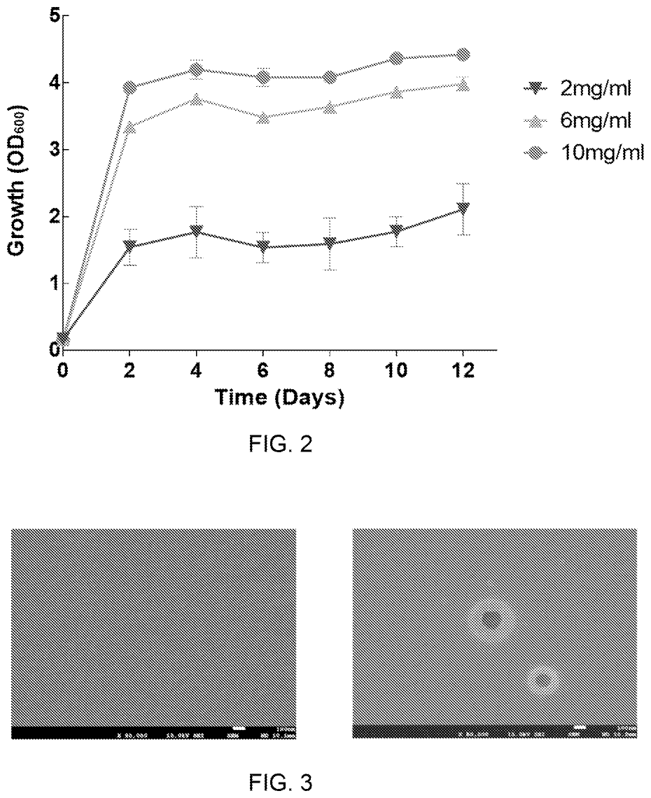

The experimental results are shown in FIG. 2.

Pseudomonas sp. JNU01 started to divide immediately after culture and reached an exponential growth phase between 1-2 days of the experiment.

Pseudomonas sp. JNU01 showed high levels of cell growth at polystyrene concentrations of 6 mg/ml or greater. After 4 days of culturing using polystyrene at a concentration of 2 g/L to 10 g/L, a stationary phase was reached.

### Experimental Example 3. Analysis to Determine Whether Polystyrene Film is Degraded by Microorganism: SEM (Scanning Electron Microscope) Analysis

(1) Experimental Method

The surface morphology of the polystyrene film was observed under a field emission scanning electron microscope (FE-SEM, Hitachi SU-70) with an acceleration voltage of 15 kV at the Korea Basic Science Institute (KBSI) in Gwangju, Jeollanam-do, Korea.

Prior to FE-SEM measurement, a conductive layer was formed directly on the surface of the polystyrene film using a platinum raw material of 20 mA for 100 seconds. This platinum coating enables FE-SEM measurement of the polystyrene polymer sample.

(2) Experimental Results: Analysis to Determine Whether Polystyrene Film is Degraded by Microorganism: SEM (Scanning Electron Microscope) Analysis

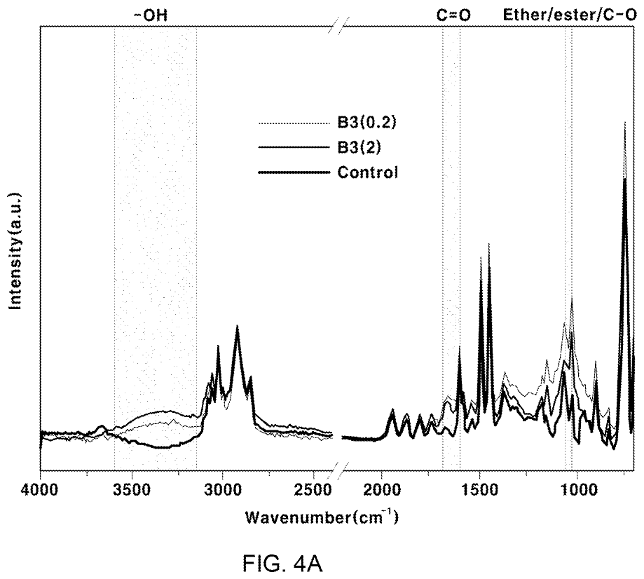

The experimental results are shown in FIG. 3.

FIG. 3 shows 50,000× FE-SEM images of the non-degraded polystyrene film and the polystyrene film biodegraded by 30 days of incubation with Pseudomonas sp. JNU01.

To remove bacteria attached to the polystyrene film, the polystyrene film was treated with a 2% sodium dodecyl sulfate (SDS) solution for 4 hours, followed by washing with distilled water and methanol.

The photograph on the left of FIG. 3 shows an SEM image of the upper outer surface of the non-degraded polystyrene film. The non-degraded polystyrene film has an intact and smooth surface without deformed portions (holes or cracks).

On the other hand, the photograph on the right of FIG. 3 shows that the polystyrene film biodegraded by Pseudomonas sp. JNU01 had a porous structure as a whole. This suggests that the microorganism biologically reacted with the polystyrene film to induce surface degradation of the polystyrene film.

After the microorganism on the surface of the polystyrene film was removed by the washing step, the microbial cells disappeared, but only the biologically formed porous structure remained. The FE-SEM images show that the polystyrene film was biodegraded by Pseudomonas sp. JNU01.

### Experimental Example 4. FT-IR Analysis to Determine Whether Polystyrene is Degraded by Microorganism

(1) Experimental Method

Fourier-transform infrared spectroscopy (FT-IR, IRAffinity-1S, Shimadzu, Kyoto, Japan) was used to measure functional groups in polystyrene samples in the wavelength range of 4,000-500 cm⁻¹ with a resolution of 4 cm⁻¹.

(2) Experimental Results: FT-IR Analysis to Determine Whether Polystyrene is Degraded by Microorganism

FT-IR measurement was performed to determine the chemical structure of the low-molecular-weight polystyrene powder that resulted from degradation by Pseudomonas sp. JNU01.

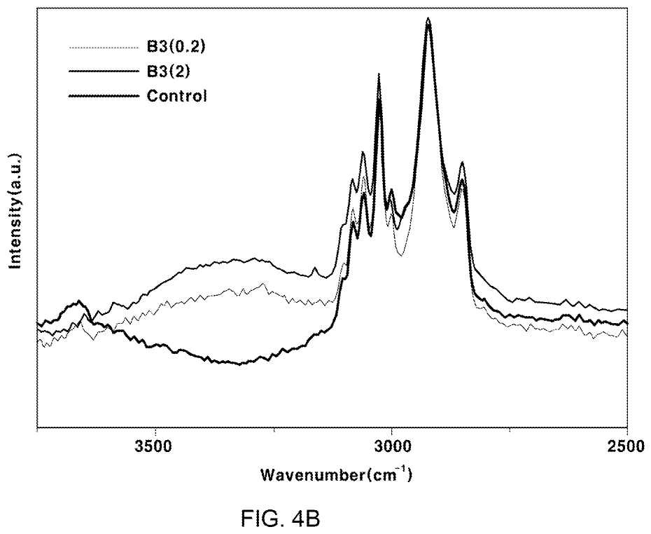

The experimental results are shown in FIGS. 4A and 4B.

FIG. 4 shows the FT-IR spectra of the non-degraded polystyrene sample (indicated as control) and the polystyrene sample microbially biodegraded by 12 days of incubation with Pseudomonas sp. JNU01.

The FR-IR spectra of the non-degraded polystyrene and the biodegraded polystyrene showed the same characteristic peaks without any noticeable change. The figure shows FT-IR spectra overlapping with the same base line for the non-degraded polystyrene and the polystyrene biodegraded by Pseudomonas sp. JNU01 at high wavenumber.

The FR-IR spectrum of the polystyrene sample biodegraded by Pseudomonas sp. JNU01 showed a weak absorption band at 3,500-3,200 cm⁻¹. This broad peak indicated —OH stretching oscillations, indicating that the polystyrene sample biodegraded by the bacteria contains hydroxyl groups.

In particular, due to Pseudomonas migulae JNU01, the FT-IR spectrum of the polystyrene sample showed very strong peak intensity for —OH stretching oscillations due to significant biological degradation. On the other hand, the FT-IR spectrum of the non-degraded polystyrene sample did not show a significant characteristic peak of —OH.

The FT-IR spectra show that the polystyrene sample may have hydroxyls (—OH) due to biological degradation by Pseudomonas migulae JNU01. This functional characteristic provides a method capable of degrading polystyrene to a lower molecular weight level than to the initial molecular weight.

What is claimed is:

1. A composition for degrading plastic comprising an isolated Pseudomonas migulae strain, or a lysate thereof, a culture, a fermentation product, or an extract thereof.

2. The composition according to claim 1, wherein the plastic comprises one or more of polyethylene terephthalate (PET), polyvinyl chloride (PVC), polystyrene (PS), polypropylene (PP) and polyethylene (PE).

3. The composition according to claim 1, wherein the strain is isolated from soil comprising plastic waste.

4. The composition according to claim 1, wherein the strain has polystyrene (PS) degradation activity.

5. The composition according to claim 1, wherein the strain has a 16S rRNA comprising the nucleotide sequence of SEQ ID NO: 1.

6. The composition according to claim 1, wherein the strain is Pseudomonas sp. JNU 01.

7. The composition according to claim 1, wherein the strain is Pseudomonas migulae JNU 01 (Accession No: KCTC 14861BP).

8. A microbial agent comprising: a microorganism comprising an isolated Pseudomonas migulae strain, a lysate thereof, a culture thereof, a fermentation product thereof, and an extract of the strain, lysate, culture or fermentation product.

9. The microbial agent according to claim 8, wherein the strain is Pseudomonas sp. JNU 01.

10. The microbial agent according to claim 8, wherein the strain is Pseudomonas migulae JNU01 (Accession No: KCTC 14861BP).

11. The microbial agent according to claim 8, wherein the microbial agent degrades a plastic.

12. The microbial agent according to claim 8, wherein the strain is one isolated from soil including a plastic and the plastic comprises polystyrene.

13. A method of degrading a plastic comprising using the composition according to claim 1, a lysate thereof, a culture thereof, a fermentation product thereof, and an extract of the strain, lysate, culture or fermentation product.

14. The method according to claim 13, wherein the plastic comprises polystyrene.

15. The method according to claim 13, wherein the strain is Pseudomonas sp. JNU 01.

16. The method according to claim 13, wherein the strain is Pseudomonas migulae JNU 01 (Accession No: KCTC 14861BP).
